(19) United States
(12) Patent Application Publication
(10) Pub. No.: US 2004/0157837 A1
(43) Pub. Date: Aug. 12, 2004

(54) Combinations for the treatment of fungal infections

(76) Inventors: George N. Serbedzija, Sudbury, MA (US); Lisa M. Johansen, Belmont, MA (US); Todd W. Chappell, Boston, MA (US); Curtis Keith, Boston, MA (US); Grant R. Zimmermann, Somerville, MA (US); M. James Nichols, Boston, MA (US); Benjamin A. Auspitz, Cambridge, MA (US)

Correspondence Address: CLARK & ELBING LLP, 101 FEDERAL STREET, BOSTON, MA, 02110, US

(21) Appl. No.: 10/703,807

(22) Filed: Nov. 7, 2003

Related U.S. Application Data
(60) Provisional application No. 60/424,657, filed on Nov. 7, 2002.

(51) Int. Cl. A61K 31/541; A61K 31/5377; A61K 31/496; A61K 31/325

(52) U.S. Cl. 514/227.8; 514/231.8; 514/252.11; 514/316; 514/478

(57) ABSTRACT

The invention features methods and compositions for treating a patient diagnosed with, or at risk for developing, a fungal infection.

### CROSS-REFERENCE TO RELATED APPLICATIONS

This application claims benefit of U.S. Provisional Application No. 60/424,657, filed Nov. 7, 2002, which is hereby incorporated by reference.

### BACKGROUND OF THE INVENTION

The invention relates to the treatment of microbial pathogens.

In animals, infections by microbial pathogens may be superficial, sub-cutaneous, or systemic. For example, fungal infections, especially of the skin and fingernails, are very common in children, although they can affect all age groups. Superficial mycoses include: tinea capitis, an infection of the neck and scalp; tinea corporis, infections of the body; tinea pedis, infections of the foot, also known as athlete's foot; tinea barbae, infections along the beard area, also known as barber's itch; tinea cruris, infections of the groin area; and tinea versicolor and tinea unguium, which involve infection of the nails. Other superficial mycoses include onychomycosis, perionychomycosis, pityriasis versicolor, oral thrush, and candidoses such as vaginal, respiratory tract, biliary, eosophageal, and urinary tract candidoses.

Systemic mycoses occur when spores are inhaled or enter into the body. Examples of systemic infections include mucocutaneous candidosis, chromoblastomycosis, mycetoma, cryptococcosis, aspergillosis, mucormycosis, paracoccidioidomycosis, North American blastomycosis, histoplasmosis, coccidioidomycosis (San Joaquin or valley fever), and sporotrichosis. As with most systemic pathogens, if left untreated, serious life-threatening infections can develop.

The need for novel antifungal treatments is significant, and is especially critical in the medical field. Immunocompromised patients provide perhaps the greatest challenge to modern health care delivery. During the last three decades there has been a dramatic increase in the frequency of fungal infections in these patients (Herbrecht, Eur. J. Haematol., 56:12, 1996; Cox et al., Curr. Opin. Infect. Dis., 6:422, 1993; Fox, ASM News, 59:515, 1993). Deep-seated mycoses are increasingly observed in patients undergoing organ transplants and in patients receiving aggressive cancer chemotherapy (Alexander et al., Drugs, 54:657, 1997). The most common pathogens associated with invasive fungal infections are the opportunistic yeast, Candida albicans, and the filamentous fungus, Aspergillus fumigatus (Bow, Br. J. Haematol., 101:1, 1998; Wamock, J. Antimicrob. Chemother., 41:95, 1998). There are an estimated 200,000 patients per year who acquire nosocomial fungal infections (Beck-Sague et al., J. Infect. Dis. 167:1247, 1993). Also adding to the increase in the numbers of fungal infections is the emergence of Acquired Immunodeficiency Syndrome (AIDS) where virtually all patients become affected with some form of mycoses during the course of the disease (Alexander et al., Drugs, 54:657, 1997; Hood et al., J. Antimicrob. Chemother., 37:71, 1996). The most common organisms encountered in these patients are Cryptococcus neoformans, Pneumocystis carinii, and C. albicans (HIV/AIDS Surveillance Report, 1996, 7(2), Year-End Edition; Polis, M. A. et al., AIDS: Biology, Diagnosis, Treatment and Prevention, fourth edition, 1997). New opportunistic fungal pathogens such as Penicillium marneffei, C. krusei, C. glabrata, Histoplasma capsulatum, and Coccidioides immitis are being reported with regularity in immunocompromised patients throughout the world.

The development of antifungal treatment regimens has been a continuing challenge. Currently available drugs for the treatment of fungal infections include amphotericin B, a macrolide polyene that interacts with fungal membrane sterols, flucytosine, a fluoropyrimidine that interferes with fungal protein and DNA biosynthesis, and a variety of azoles (e.g., ketoconazole, itraconazole, and fluconazole) that inhibit fungal membrane-sterol biosynthesis (Alexander et al., Drugs, 54:657, 1997). Even though amphotericin B has a broad range of activity and is viewed as the “gold standard” of antifungal therapy, its use is limited due to infusion-related reactions and nephrotoxicity (Wamock, J. Antimicrob. Chemother., 41:95, 1998). Flucytosine usage is also limited due to the development of resistant microbes and its narrow spectrum of activity. The widespread use of azoles is causing the emergence of clinically-resistant strains of Candida spp.

### SUMMARY OF THE INVENTION

We have discovered that the combination of an antifungal drug, amphotericin B, and a dithiocarbamoyl disulfide, disulfiram, brings about substantial inhibition of growth of triazole-resistant and triazole-susceptible strains of Candida albicans in vitro. Furthermore, this amphotericin/disulfiram combination was also found to be effective against another Candida species (C. glabrata) and against Aspergillus fumagatus. Thus, this combination can be used to treat fungal infections caused by strains of C. albicans, C. glabrata, or A. fumagatus, among others. Moreover, based on the shared action among different antifungal agents, amphotericin can be replaced by a different antifungal agent in the combination. Likewise, based on the shared action among different dithiocarbamoyl disulfide family members, disulfiram can be replaced by a different dithiocarbamoyl disulfide in the combination.

Accordingly, in one aspect, the invention features a method for treating a patient having a fungal infection by administering to the patient: (i) a first compound having a formula (I), (II), or (III), or pharmaceutically acceptable salts thereof and (ii) an antifungal agent.

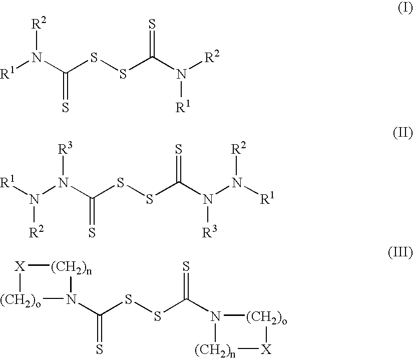

In any of these formulae, X is CH₂, O, S, NR⁴, N(CH₂)ₚOR⁵, CH(CH₂)_{q}OR⁶, CH(CH₂)ᵣCO₂R⁷, CH(CH₂)ₙCONR⁸R⁹,

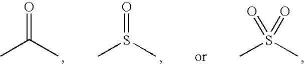

where R¹ and R² are independently C₁-C₈ linear or branched alkyl (preferably C₁-C₄ linear or branched alkyl), alkaryl, or aryl, R³, R⁴, R⁵, R⁶, R⁷, R⁸, and R⁹ are independently H, C₁-C₈ linear or branched alkyl (preferably C₁-C₄ linear or branched alkyl), alkaryl, or aryl, n is 0-3, o is 2-4, p is 2-6, and q, r, and s is 0-6. The first compound and the antifungal agent are administered within 10 days of each other, within five days of each other, within 24 hours of each other, within one hour of each other, or are administered simultaneously, in amounts sufficient to treat a fungal infection in the patient.

In a related aspect, the invention features a method for treating a patient who has a fungal infection, or inhibiting the development of a fungal infection in a patient who is at risk for developing a fungal infection by administering to the patient a compound of formula (I), (II), or (III) and an antifungal agent. The compound of formula (I), (II), or (III) and the antifungal agent are administered within 10 days of each other, within five days of each other, within 24 hours of each other, within one hour of each other, or are administered simultaneously, in amounts sufficient to treat or inhibiting the development of a fungal infection in the patient.

Fungal infections treated according to the invention can cause, for example, the following conditions: oral thrush, vaginal candidosis, respiratory tract candidosis, biliary candidosis, eosophageal candidosis, urinary tract candidosis, systemic candidosis, and mucocutaneous candidosis.

In another aspect, the invention features a method of preventing, stabilizing, or inhibiting the growth of fungal cells. This method includes the steps of contacting fungal cells with a compound of formula (I), (II), or (III), and an antifungal agent in amounts that, in combination, are sufficient to prevent, stabilize, or inhibit the growth of fungal cells.

In a related aspect, the invention features a method for preventing, stabilizing, or inhibiting the growth of fungal cells on a surface. This method includes the steps of contacting the surface with an amount of a combination of a compound of formula (I), (II), or (III) and an antifungal agent sufficient to prevent stabilize, or inhibit growth of fungal cells.

Exemplary surfaces include process equipment, water sanitation systems, cooking utensils, food preparation areas, and medical devices such as, surgical and dental tools, dental appliances including dentures, oral rinses, stents, endoscopy equipment, surgical implants, prosthetic devices, artificial joints, heart valves, pacemakers, vascular grafts, vascular catheters, cerebrospinal fluid shunts, urinary catheters, and continuous ambulatory peritoneal dialysis catheters.

In another aspect, the invention features a pharmaceutical composition that includes a compound of formula (I), (II), or (III), an antifungal agent, and a pharmaceutically acceptable carrier. The compound of formula (I), (II), or (III), and antifungal agent are each present in amounts that, when administered together to a patient, inhibit or reduce fungal growth.

In a related aspect, the invention features a pharmaceutical pack containing a compound of formula (I), (II), or (III), and an antifungal agent.

The compound of formula (I), (II), or (III) is administered or prepared for administration in an amount between 0.001 and 500 mg per day, desirably, between 25 and 250 mg per day. The antifungal agent is administered in an amount between 0.001 and 2000 mg per day, desirably, between 0.5 and 1000 mg per day. The compound of formula (I), (II), or (III), and antifungal agent may be administered at a ratio of 0.1-1000 parts, by molar ratio, of compound of formula (I), (II), or (III) to one part, by molar ratio, of antifungal agent. Formulation and administration of the compound of formula (I), (II), or (III), and antifungal agent can be prepared for intravenous, intramuscular, transdermal, rectal, oral, topical, intravaginal, ophthalmic, or inhalation administration.

In yet another aspect, the invention features a method for identifying combinations of compounds useful for treating a patient having a fungal infection. This method includes the steps: (a) contacting fungal cells or cells infected with a fungus in vitro with (i) an antifungal agent or a compound of formula (I), (II), or (III) and (ii) a candidate compound; and (b) determining whether the combination of antifungal agent, and the candidate compound of formula (I), (II), or (III) reduces growth of fungal cells relative to fungal cells contacted with the antifungal agent or compound of formula (I), (II), or (III) but not contacted with the candidate compound, or fungal cells contacted with the candidate compound but not with the antifungal agent or compound of formula (I), (II), or (III). A decrease in fungal cell growth identifies a combination as a combination that is useful for treating a patient having a fungal infection.

Desirably, the antifungal agent is chosen from amphotericin B, amorolfine, anidulafungin, butenafine, butoconazole, candidin, carbol-fuchsin, caspofungin, ciclopirox, clotrimazole, dapsone, econazole, enilconazole, fluconazole, flucytosine, gentian violet, griseofulvin, haloprogin, itraconazole, ketoconazole, mafenide, micafungin, miconazole, naftifine, nystatin, oxiconazole, pimaricin, posaconazole, ravoconazole, rimocidin, silver sulfadiazine, sulconazole, terbinafine, terconazole, tioconazole, tolnaftate, undecylenic acid, vacidin A, and voriconazole, while the compound of formula (I), (II), or (III) is chosen from: disulfiram (bis(diethylthiocarbamoyl) disulfide), bis(dimethylthiocarbamoyl) disulfide, bis(dipropylthiocarbamoyl) disulfide, bis(dibutylthiocarbamoyl) disulfide, bis(dipentylthiocarbamoyl) disulfide, bis(di(2-methylpropyl)thiocarbamoyl) disulfide, bis(piperidinothiocarbamoyl) disulfide, bis(morpholinothiocarbamoyl) disulfide, bis((4-methylpiperazino)thiocarbamoyl) disulfide, bis((4-(2-hydroxyethyl)piperazino)thiocarbamoyl) disulfide, bis((hexahydro-4-methyl-1H-1,4-diazepin-1-yl)thiocarbamoyl) disulfide, and bis(3,3-dimethylcarbazoyl) disulfide.

Treatment with the aforementioned antifungal compositions and combinations are directed to the following: Absidia corymbifera, Acremonium falciforme, A. kiliense, A. recifei, Ajellomyces dermatitidis, A. capsulata, Aspergillus spp., (e.g., A. flavus, A. fumigatus, A. nidulans, A. niger, A. terreus), Candida spp. (e.g., C. albicans, C. glabrata, C. guillermondii, C. krusei, C. parapsilosis, C. kefyr, C. tropicalis), Cryptococcus neoformans, Cunninghamella elegans, Emmonsia parva, Epidermophyton floccosum, Exophialia dermitidis, E. werneckii, E. jeanselmei, E. spinifera, E. richardsiae, Filobasidiella neoformans, Fonsecaea compacta, F. pedrosoi, Histoplasma capsulatum, Leptoshaeria senegarlensis, Madurella mycetomatis, M. grisea, Malassezia furfur, Microsporum spp, Neotestudina rosatii, Paracoccidioides brasiliensis, Penicillium marneffei, Phialophora verrucosa, Piedraia hortae, Pneumocystis carinii, Pseudallescheria boydii, Pyrenochaeta romeroi, Rhizomucor pusillus, Sporothrix schenckii, Trichophyton spp, Trichosporon beigelii, Wangiella dermatitidis and Xylohypha bantiana.

Fungal infections treated according to the invention can cause the following conditions: tinea capitis, tinea corporis, tinea pedis, tinea barbae, tinea cruris, tinea versicolor, onychomycosis, perionychomycosis, pityriasis versicolor, tinea unguium, oral thrush, vaginal candidosis, respiratory tract candidosis, biliary candidosis, eosophageal candidosis, urinary tract candidosis, systemic candidosis, mucocutaneous candidosis, mycetoma, cryptococcosis, aspergillosis, mucormycosis, chromoblastomycosis, paracoccidioidomycosis, North American blastomycosis, histoplasmosis, coccidioidomycosis, or sporotrichosis.

By “antifungal agent” is meant an agent that reduces or inhibits the growth of a fungus by at least 10%, relative to an untreated control, with the proviso that the agent does not belong to the dithiocarbamoyl disulfide class of compounds. Exemplary antifungal agents are provided herein.

By “fungal infection” is meant an invasion of a host animal by fungal cells. For example, the infection may include the excessive growth of fungi that are normally present in or on the animal, or growth of fungi that are not normally present in or on the animal. More generally, a fungal infection can be any situation in which the presence of a fungal population is detrimental or damaging to a host animal. In one embodiment, the number of a particular genus or species of fungus is at least 2, 4, 6, or 8 times the number normally found in the animal.

By “an effective amount” is meant an amount of a compound, in a combination of the invention, required to treat or prevent a fungal infection. The effective amount of active compound(s) used to practice the present invention for therapeutic or prophylactic treatment of conditions caused by or contributed to by a fungal infection varies depending upon the manner of administration, the age, body weight, and general health of the subject. Ultimately, the attending physician or veterinarian will decide the appropriate amount and dosage regimen. Such amount is referred to as an “effective” amount.

By “low dosage” is meant a dosage of an agent that is less than the dosage prescribed when that agent is used alone. For example, a low dosage of systemic amphoterecin B is a dosage of less than 3 mg/kg body weight. A low dosage of fluconazole is less than 100 mg/dosage.

As used herein, the terms “alkyl,” “alkenyl,” and the prefix “alk-” are inclusive of both straight chain and branched chain groups and of cyclic groups, i.e., cycloalkyl and cycloalkenyl groups. Cyclic groups can be monocyclic or polycyclic and preferably have from 3 to 10 ring carbon atoms, inclusive. Exemplary cyclic groups include cyclopropyl, cyclopentyl, cyclohexyl, and adamantyl groups.

By “aromatic residue” is meant an aromatic group having a ring system with conjugated π electrons (e.g., phenyl, or imidazole). The ring of the aryl group preferably has 5 to 10 atoms. The aromatic ring may be exclusively composed of carbon atoms or may be composed of a mixture of carbon atoms and heteroatoms (i.e., nitrogen, oxygen, sulfur, and phosphorous). Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

The term “aryl” means mono or bicyclic aromatic or heteroaromatic rings or ring systems. Examples of aryl groups include phenyl, naphthyl, pyrrolyl, furanyl, indolyl, benzofuranyl, benzothiophenyl, imidazolyl, triazolyl, tetrazolyl, benzimidazolyl, oxazolyl, benzoxazolyl, thiazolyl, benzothiazolyl, pyrazolyl, benzopyrazolyl, isoxazolyl, benzisoxazolyl, isothiazolyl, benzisothiazolyl, pyridinyl, quinolinyl, and isoquinolinyl.

“Heterocyclyl” means non-aromatic rings or ring systems that contain at least one ring hetero atom (e.g., O, S, N, P). Heterocyclic groups include, for example, pyrrolidinyl, tetrahydrofuranyl, morpholinyl, thiazolidinyl, and imidazolidinyl groups.

Aryl and heterocyclyl groups may be unsubstituted or substituted by one or more substituents selected from the group consisting of C₁₋₁₀ alkyl, hydroxy, halo, nitro, C₁₋₁₀ alkoxy, C₁₋₁₀ alkylthio, trihalomethyl, C₁₋₁₀ acyl, arylcarbonyl, heteroarylcarbonyl, nitrile, C₁₋₁₀ alkoxycarbonyl, oxo, arylalkyl (wherein the alkyl group has from 1 to 10 carbon atoms) and heteroarylalkyl (wherein the alkyl group has from 1 to 10 carbon atoms).

Compounds useful in the invention include those described herein in any of their pharmaceutically acceptable forms, including racemic mixtures and substantially pure isomers (e.g., diastereomers, enantiomers) of compounds described herein, as well as salts, solvates, and polymorphs thereof.

The combinations of the invention are used to treat microbial pathogen infections. That the compounds work together means that lower doses of each can be used, decreasing the possibility of toxicity. For example, the combination of a compound of formula (I), (II), or (III) and an antifungal agent for the treatment of fungal infections, both used at dosages that are standard for each, exhibits greater therapeutic efficacy than each administered alone.

Other features and advantages of the invention will be apparent from the following detailed description, and from the claims.

### DETAILED DESCRIPTION

We have discovered that the combination of an antifungal drug, amphotericin B, and a dithiocarbamoyl disulfide, disulfiram, has antifungal activity above that of either amphotericin B or disulfiram alone. Thus, combinations of disulfiram and amphotericin B are useful for the treatment of fungal infections. In addition, the ability to use these two agents in combination has potential for the mitigation of side effects that could be encountered by the use of amphotericin B alone at high doses.

Amphotericin B

Amphotericin B is a polyene antibiotic isolated from Streptomyces nodosus. It contains a macrolide ring and an aminosugar, mycosamine. The formula of amphotericin B is:

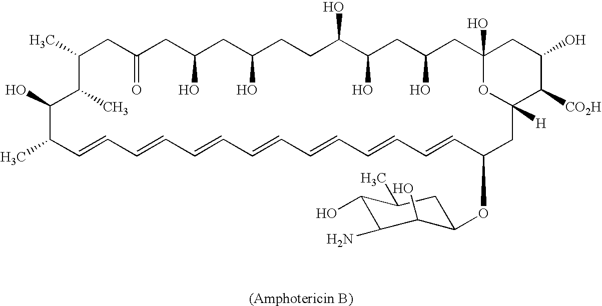

Amphotericin B is currently used for a wide range of systemic fungal infections and is formulated for IV injection and administered in this manner or intrathecally. It is poorly water soluble, but is sufficiently soluble that it is administered by IV infusion (0.1 mg/mL) or (0.3 mg/mL) in 5% dextrose. It is unstable in solution, particularly in normal saline. Other polyene macrolide antifungal agents include nystatin, candidin, rimocidin, vacidin A, and pimaricin.

Other Antifungal Agents

Antifungal agents are known that derive their mechanism of action by their inhibition of cytochrome-P450 activity, which decreases conversion of 14-alpha-methylsterols to ergosterol. Failure of ergosterol synthesis causes altered membrane permeability leading to loss of ability to maintain a normal intracellular environment. Examples of antifungal agents that inhibit ergosterol biosynthesis by their cytochrome-P450 activity are fluconazole, itraconazole, ketoconazole, clotrimazole, butoconazole, econazole, ravuconazole, oxiconazole, posaconazole, sulconazole, terconazole, tioconazole, and voriconazole. Other antifungal agents that are egosterol biosynthesis inhibitors act by blocking squalene epoxidation. Examples of antifungal agents that inhibit ergosterol biosynthesis by blocking squalene epoxidation are amorolfine, butenafine, naftifine, and terbinafine.

Flucytosine is an antifungal agent that is known to derive its mechanism of action by its antimetabolic activity. It is converted to 5-fluorouracil (5-FU), which inhibits thymidylate synthetase and thereby inhibits fungal protein synthesis.

Griseofulvin is an antifungal agent that inhibits fungal mitosis by disrupting the mitotic spindle through its interaction with polymerized microtubules.

Antifungal agents are also known that serve as glucan synthesis inhibitors. Glucan is a key component of the fungal cell wall, and inhibition of this enzyme produces significant antifungal effects. Examples of glucan synthesis inhibitors are caspofungin, micafungin, and anidulafungin.

Disulfiram, or another dithiocarbamoyl disulfide, may be used in combination with any of the foregoing antifungal agents such that the dose of the antifungal agent is lowered and any side effects resulting from its mechanism of action mitigated.

Dithiocarbamoyl Disulfides

Disulfiram [bis(diethylthiocarbamoyl) disulfide] is a member of the dithiocarbamoyl disulfide class of compounds. It occurs as a white to off-white, odorless, and almost tasteless powder, soluble in water to the extent of about 20 mg/100 mL, and in alcohol to the extent of about 3.8 mg/100 mL. It is currently formulated for oral administration, with each tablet containing 250 mg or 500 mg of disulfiram. Its formula is:

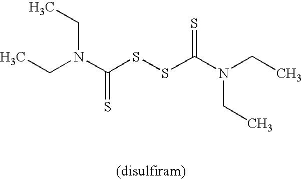

Some analogs of disulfiram have the following formulae:

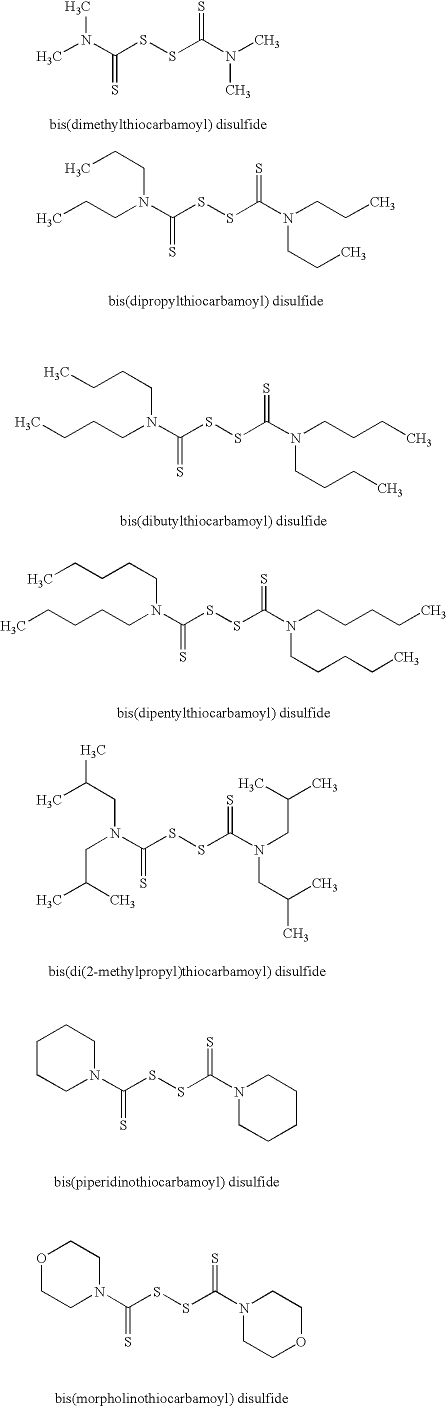

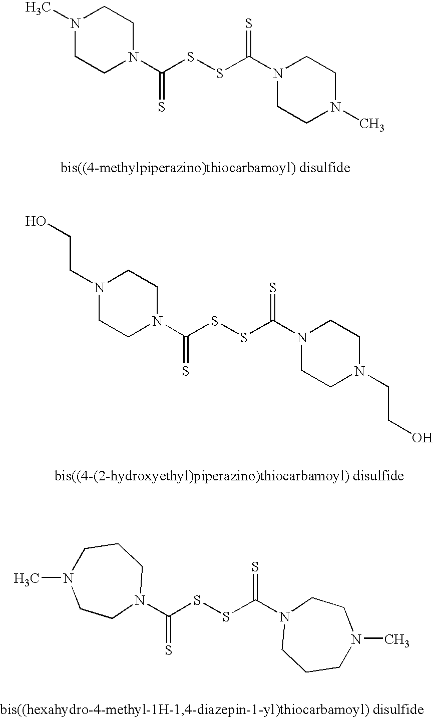

Preferred analogs are described, for example, by formulae (I), (II), or (III):

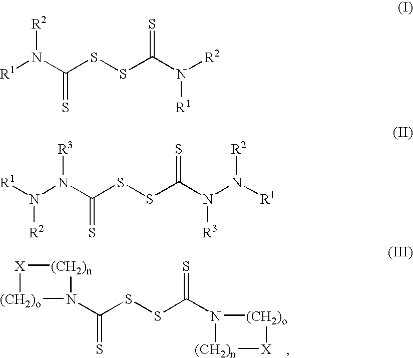

wherein X is CH₂, O, S, NR⁴, N(CH₂)ₚOR⁵, CH(CH₂)_{q}OR⁶, CH(CH₂)ᵣCO₂R⁷, CH(CH₂)ₛCONR⁸R⁹,

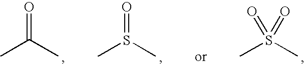

where R¹ and R² are independently C₁-C₈ linear or branched alkyl, alkaryl, or aryl, R³, R⁴, R⁵, R⁶, R⁷, R⁸, and R⁹ are independently H, C₁-C₈ linear or branched alkyl, alkaryl, or aryl, n is 0-3, o is 2-4, p is 2-6, and q, r, or s is 0-6.

Pharmaceutically acceptable salts of disulfiram and related dithiocarbamoyl disulfides are also useful compounds of the invention, as are metal chelates of these compounds. Preferred metals include, for example, copper, manganese, iron, and zinc.

For oral administration of disulfiram, the recommended adult dosage is 250 mg daily, with a maximum dosage of 500 mg daily.

Therapy

Combination therapy according to the invention may be performed alone or in conjunction with another therapy and may be provided at home, the doctor's office, a clinic, a hospital's outpatient department, or a hospital. Treatment generally begins at a hospital so that the doctor can observe the therapy's effects closely and make any adjustments that are needed. The duration of the combination therapy depends on the age and condition of the patient, the stage of the patient's disease, and how the patient responds to the treatment. Additionally, a person having a greater risk of developing a fungal infection (e.g., a person who is to undergo a surgical procedure) may receive prophylactic treatment to inhibit or delay the onset of symptoms.

The dosage, frequency and mode of administration of each component of the combination can be controlled independently. For example, one compound may be administered orally three times per day, while the second compound may be administered topically once per day. Combination therapy may be given in on-and-off cycles that include rest periods so that the patient's body has a chance to recovery from any as yet unforeseen side-effects. The compounds may also be formulated together such that one administration delivers both compounds.

Formulation of Pharmaceutical Compositions

The administration of each compound of the combination may be by any suitable means that results in a concentration of the compound that, combined with the other component, is anti-fungal upon reaching the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalent, skin (patch), or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, or aerosols. The pharmaceutical compositions may be formulated according to conventional pharmaceutical practice (see, e.g., Remington: The Science and Practice of Pharmacy (20th ed.), ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York).

Dosages

The dosage of each compound of the claimed combinations depends on several factors, including: the administration method, the disease to be treated, the severity of the disease, whether the disease is to be treated or prevented, and the age, weight, and health of the person to be treated.

The compound of formula (I), (II), or (III) is preferably administered or prepared for administration in an amount of about 0.001-500 mg per day, and more preferably in an amount of about 25-250 mg per day. The antifungal agent is preferably administered or prepared for administration in an amount of about 0.001-2000 mg per day, and more preferably in an amount of about 25-1000 mg per day. Each component of the combination is normally administered one to ten times daily (preferably one to five times daily, more desirably one or two times daily).

Other Uses

Combinations of the invention may also be used for the preservation of food, beverages, cosmetics such as lotions, creams, gels, ointments, soaps, shampoos, conditioners, antiperspirants, deodorants, mouthwash, contact lens products, enzyme formulations, or food ingredients. Combinations of the invention can be incorporated into, for example, unpreserved food, beverages, cosmetics, contact lens products, or food ingredients in an amount effective for killing or inhibiting the growth of fungi.

Thus, a combination of the invention may be useful as a disinfectant, e.g., in the treatment of acne, eye infections, mouth infections, fingernail infections, toenail infections, skin infections, wounds, or in treating infections caused by the insertion of stents. Combinations of the invention are also useful for cleaning, disinfecting, or inhibiting fungal growth on any hard surface. Examples of surfaces which may advantageously be contacted with a combination of the invention are surfaces of process equipment used in dairies, chemical or pharmaceutical process plants, water sanitation systems, paper pulp processing plants, water treatment plants, cooling towers, cooking utensils, or surfaces in any area in which food is prepared (e.g., hospitals, nursing homes, or restaurants).

In addition, combinations of the invention are useful for cleaning, disinfecting, or inhibiting fungal growth on or in an in-dwelling device in a patient. In-dwelling devices include, but are not limited to, surgical and dental implants, prosthetic devices, artificial joints, heart valves, pacemakers, vascular grafts, vascular catheters, stents, cerebrospinal fluid shunts, urinary catheters, and continuous ambulatory peritoneal dialysis (CAPD) catheters. A combination of the invention may be used to bathe an in-dwelling device immediately before insertion. Alternatively, the combination may be administered by injection to achieve a local or systemic effect against relevant fungi shortly before insertion of an in-dwelling device. Treatment may be continued after surgery during the in-body time of the device.

### EXAMPLE

The following example is to illustrate the invention. It is not meant to limit the invention in any way.

Using the method described below, we tested the ability of the combination of various concentrations of amphotericin B plus disulfiram to inhibit the proliferation of numerous yeast strains using particle density as an indicator of cell number.

From the anti-proliferation experiments in yeast, we observed that for C. albicans strain 17 that 0.36 μM of amphotericin generated approximately 50% inhibition of growth (Table 1). When moderate concentrations (0.5 μM) of disulfiram were combined with the same concentration of amphotericin we observed approximate 90% inhibition of growth. We obtained similar dose shifting of amphotericin in anti-proliferation experiments with other yeast strains, C. albicans MYA 573 (Table 2), C. albicans strain 1 (Table 3), and C. glabrata (Table 4). Anti-proliferation experiments in Aspergillus fumagatus (Table 5) generated does shifting but at slightly higher doses.

TABLE 1
Percent inhibition of proliferation of
Candida albicans Strain 17

| | Disulfiram (μM) | | | | | | | | | | |
|---|---|---|---|---|---|---|---|---|---|---|---|
| | | 1.12 | 0.56 | 0.28 | 0.14 | 0.07 | 0.04 | 0.02 | 0.01 | 0.00 | 0.00 |
| Amphotericin | 0.72 | 98.73 | 98.70 | 98.40 | 95.22 | 93.02 | 97.66 | 97.23 | 97.82 | 97.58 | 72.18 |
| B (μM) | 0.36 | 94.99 | 91.68 | 89.34 | 85.64 | 83.72 | 84.90 | 85.66 | 82.98 | 83.69 | 51.08 |
| | 0.18 | 70.40 | 68.58 | 62.46 | 59.97 | 43.95 | 56.49 | 56.29 | 54.10 | 49.60 | 43.28 |
| | 0.09 | 63.70 | 45.38 | 42.70 | 28.36 | 31.68 | 24.51 | 23.20 | 22.53 | 23.80 | 16.03 |
| | 0.05 | 62.99 | 28.92 | 12.00 | 7.74 | 3.38 | 7.63 | 10.38 | 3.97 | 1.61 | −1.91 |
| | 0.02 | 45.66 | 11.39 | 6.47 | 1.43 | −8.49 | −0.94 | −0.91 | −2.59 | −3.43 | −8.29 |
| | 0.01 | 63.18 | 41.38 | 15.07 | 3.51 | −13.19 | 4.12 | 7.39 | −2.62 | −1.86 | −4.61 |
| | 0.01 | 53.50 | 31.34 | 15.22 | 4.24 | 4.07 | 8.56 | 9.23 | 5.38 | −3.00 | −6.27 |
| | 0.00 | 52.81 | 28.99 | 7.66 | 14.45 | 0.43 | −1.83 | 3.14 | −6.13 | 0.35 | −9.25 |
| | 0.00 | 53.44 | 30.53 | 22.87 | 2.66 | 2.27 | −0.15 | −0.03 | −3.64 | −4.50 | −10.33 |

TABLE 2
Percent inhibition of proliferation of
Candida albicans MYA 573

| | Disulfiram (μM) | | | | | | | | | | |
|---|---|---|---|---|---|---|---|---|---|---|---|
| | | 1.12 | 0.56 | 0.28 | 0.14 | 0.07 | 0.04 | 0.02 | 0.01 | 0.00 | 0.00 |
| Amphotericin | 0.72 | 98.77 | 98.66 | 98.28 | 96.59 | 92.83 | 91.42 | 92.46 | 94.95 | 95.32 | 95.75 |
| B (μM) | 0.36 | 96.48 | 93.36 | 88.61 | 74.94 | 79.64 | 69.68 | 67.53 | 68.97 | 69.38 | 62.35 |
| | 0.18 | 91.94 | 90.70 | 70.96 | 68.43 | 57.41 | 42.59 | 41.36 | 38.87 | 34.40 | 41.95 |
| | 0.09 | 94.25 | 82.21 | 73.86 | 58.67 | 44.85 | 36.38 | 22.77 | 14.29 | 20.09 | 4.99 |
| | 0.05 | 94.69 | 75.69 | 50.07 | 38.00 | 21.88 | 17.85 | 4.57 | 2.47 | 0.71 | 11.55 |
| | 0.02 | 93.93 | 77.94 | 63.06 | 37.14 | 21.85 | 8.69 | 11.55 | 6.85 | −4.54 | −2.37 |
| | 0.01 | 96.81 | 88.47 | 44.19 | 35.26 | 15.06 | 8.89 | 1.26 | −4.92 | −5.63 | −1.32 |
| | 0.01 | 94.33 | 82.26 | 54.86 | 22.90 | 21.58 | 13.50 | 0.14 | −8.03 | −5.31 | −3.02 |
| | 0.00 | 91.81 | 74.47 | 61.31 | 25.41 | 16.82 | 4.79 | 3.70 | −0.54 | −4.61 | −8.52 |
| | 0.00 | 95.01 | 78.35 | 55.59 | 39.26 | 25.17 | 10.30 | 1.70 | −5.62 | −9.43 | 0.89 |

TABLE 3
Percent inhibition of proliferation of
Candida albicans Strain 1

| | Disulfiram (μM) | | | | | | | | | | |
|---|---|---|---|---|---|---|---|---|---|---|---|
| | | 1.12 | 0.56 | 0.28 | 0.14 | 0.07 | 0.04 | 0.02 | 0.01 | 0.00 | 0.00 |
| Amphotericin | 0.72 | 97.86 | 98.54 | 98.59 | 93.45 | 91.47 | 98.09 | 98.33 | 97.57 | 97.96 | 97.82 |
| B (μM) | 0.36 | 95.60 | 94.56 | 91.97 | 89.39 | 85.28 | 90.62 | 92.49 | 94.01 | 87.64 | 86.21 |
| | 0.18 | 92.31 | 83.01 | 69.46 | 62.76 | 59.26 | 54.82 | 59.83 | 53.72 | 56.22 | 45.73 |
| | 0.09 | 88.24 | 73.85 | 38.84 | 29.96 | 32.28 | 22.46 | 16.21 | 15.41 | 17.54 | 15.87 |
| | 0.05 | 81.38 | 71.77 | 19.58 | 16.47 | 5.44 | 3.24 | 11.15 | −1.01 | 5.98 | 6.68 |
| | 0.02 | 87.26 | 58.73 | 30.40 | 14.42 | 10.77 | 4.65 | 15.03 | 6.78 | 0.40 | 0.07 |
| | 0.01 | 91.47 | 64.32 | 38.65 | 16.02 | 5.03 | −5.44 | 2.09 | 1.49 | −6.17 | −2.05 |
| | 0.01 | 89.75 | 61.74 | 31.84 | 22.01 | 7.70 | 3.87 | −0.65 | −8.43 | −2.50 | −12.39 |
| | 0.00 | 89.42 | 59.21 | 25.66 | 13.39 | 11.52 | 12.96 | 1.24 | 0.70 | 2.57 | −7.43 |
| | 0.00 | 89.64 | 54.54 | 26.85 | 18.23 | 11.66 | 10.08 | 9.08 | 5.30 | 3.61 | −4.73 |

TABLE 4
Percent inhibition of proliferation of Candida glabrata

| | Disulfiram (μM) | | | | | | | | | | |
|---|---|---|---|---|---|---|---|---|---|---|---|
| | | 1.12 | 0.56 | 0.28 | 0.14 | 0.07 | 0.04 | 0.02 | 0.01 | 0.00 | 0.00 |
| Amphotericin | 0.72 | 91.61 | 97.55 | 97.90 | 91.29 | 91.35 | 97.81 | 98.00 | 97.82 | 97.85 | 98.01 |
| B (μM) | 0.36 | 94.71 | 93.33 | 90.78 | 83.39 | 81.08 | 88.07 | 90.12 | 90.52 | 87.56 | 83.84 |
| | 0.18 | 88.01 | 77.40 | 56.27 | 57.37 | 51.01 | 51.22 | 52.59 | 47.18 | 44.66 | 38.76 |
| | 0.09 | 86.84 | 72.02 | 27.53 | 24.88 | 22.52 | 20.10 | 10.93 | 4.69 | 13.90 | 18.18 |
| | 0.05 | 81.93 | 59.45 | 27.78 | 17.78 | 16.02 | 11.70 | 0.82 | −5.15 | 1.69 | −13.33 |
| | 0.02 | 86.96 | 58.08 | 20.67 | 9.87 | 8.18 | 14.75 | 10.94 | −3.50 | −4.76 | −7.49 |
| | 0.01 | 86.83 | 60.28 | 31.80 | 30.44 | 1.33 | 14.24 | 15.63 | 11.27 | 1.10 | −6.52 |
| | 0.01 | 89.40 | 67.67 | 19.35 | 9.35 | 1.27 | 11.64 | −0.45 | 5.25 | 0.57 | −1.59 |
| | 0.00 | 83.00 | 53.83 | 24.88 | 5.30 | 3.43 | 15.27 | −3.79 | 2.44 | −1.42 | −7.99 |
| | 0.00 | 88.66 | 61.32 | 19.60 | 2.12 | 1.92 | 1.52 | −1.67 | −10.43 | −3.43 | −5.73 |

TABLE 5
Percent inhibition of proliferation of Aspergillus fumigatus

| | Disulfiram (μM) | | | | | | | | | | |
|---|---|---|---|---|---|---|---|---|---|---|---|
| | | 10.00 | 5.00 | 2.50 | 1.25 | 0.63 | 0.31 | 0.16 | 0.08 | 0.04 | 0.00 |
| Amphotericin | 2.00 | 94.52 | 94.47 | 95.01 | 91.44 | 90.51 | 92.27 | 91.04 | 94.37 | 93.55 | 93.70 |
| B (μM) | 1.00 | 95.12 | 94.92 | 95.86 | 91.90 | 90.21 | 92.79 | 89.17 | 94.28 | 93.21 | 94.42 |
| | 0.50 | 95.02 | 94.96 | 94.39 | 91.14 | 89.96 | 91.76 | 91.14 | 93.46 | 92.55 | 95.09 |
| | 0.25 | 94.06 | 94.39 | 93.82 | 92.01 | 89.24 | 91.53 | 90.46 | 94.97 | 90.84 | 89.09 |
| | 0.13 | 92.81 | 92.65 | 93.90 | 79.44 | 53.05 | 61.51 | 66.47 | 66.38 | 40.58 | 44.38 |
| | 0.06 | 56.50 | 42.75 | 55.39 | 19.58 | −9.30 | 1.77 | 2.53 | 12.79 | 12.75 | 2.80 |
| | 0.03 | 49.11 | 26.13 | 44.49 | 2.30 | 2.84 | −8.87 | −23.77 | 12.60 | −14.83 | −4.97 |
| | 0.02 | 39.75 | 29.29 | 12.83 | −7.62 | 2.75 | 5.25 | 11.38 | 9.12 | 0.23 | 10.70 |
| | 0.01 | 32.50 | 27.50 | 10.54 | −1.84 | −6.99 | −9.50 | 6.34 | 0.89 | 13.85 | 3.10 |
| | 0.00 | 37.21 | 31.73 | 14.88 | 6.07 | −0.75 | −13.08 | −2.21 | −2.93 | 0.66 | 14.10 |

Experimental Procedures

The foregoing results were obtained with the following materials and methods.

Candida Strains

Two clinical Candida albicans isolates (one triazole-resistant strain and one triazole-susceptible strain) were obtained from the culture collection if the Seattle Biomedical Research Institute (Seattle, Wash.). Additional fungal strains were obtained from the American Type Culture Collection triazole-resistant C. albicans: ATCC MYA-573, C. glabrata: ATCC 90030. Yeast cultures were stored in 15% glycerol at −70° C. Isolates were cultured in growth medium (RPMI 1640; 2% glucose without NaHCO₃ and phenol red, buffered with 0.165 M MOPS to pH 7.0) at 35° C. for 24 hours prior to in vitro susceptibility testing. All media reagents were purchased from Sigma Chemical Co. (St. Louis, Mo.).

Aspergillus Strain

The Aspergillus fumigatus strain was obtained from the American Type Culture Collection (ATCC 46645). Aspergillus cultures were stored in 15% glycerol at −70° C. Isolates were cultured on Sabouraud glucose (SAB) agar plates at 37° C. for 5 to 7 days. Condidia were collected by addung 5 to 10 mL of sterile saline containing 0.05% Tween 20 to the plates and loosening condidia with a swab. Using a pipette, condidia were collected suspension and placed in a sterile bottle. This was repeated for all plates. After harvesting the suspension was filtered through gauze into a clean, sterile bottle. The turbidity of the supernatants was measured spectrophometrically at 530 nm and the transmission adjusted to 80-82% with PBS (with 0.05% Tween 20) to yield an initial suspension of 1×10⁶ CFU/mL.

Compounds and Compound Preparation

The following compounds were used: disulfiram and amphotericin B (both from Sigma Chemical Co.). In the anti-proliferation experiments, Gentian Violet was used as a positive control for antifungal activity. Stock solutions of each compound were prepared in DMSO and stored at −20° C. Prior to use, stock solutions were diluted in RPMI to produce 10× solutions. 10× disulfiram (0-7 μL) and 10× amphotericin B (0-7 μL) were then plated in 40 μL of in 384 well microtiter plates to form a matrix of compound combinations.

Analogs of disulfiram are prepared in a manner similar to the synthesis of disulfiram, whose synthesis is as follows: A solution of diethylamine (10.0 g, 0.137 mol) in 100 mL of water is treated with 13.7 mL of 1.0M NaOH. Carbon disulfide (8.24 mL, 0.137 mol) is added dropwise and the solution was stirred for 1 hr. The solution is cooled with an ice bath and 15.5 mL of 30% hydrogen peroxide is added dropwise while keeping the temperature of the reaction mixture below 10° C. The reaction mixture is stirred for 4 hours at 0° C. and the resulting off-white solid is filtered and collected to yield disulfiram (35.0 g, 86% yield).

Anti-Proliferation Assay

All antifungal susceptibility testing was performed according to document M-27A as published by the National Committee for Clinical Laboratory Standards (Reference Method for Broth Dilution Antifungal Susceptibility Testing of Yeasts (1997), Wayne, Pa.). Briefly, yeast inocula from overnight cultures were standardized to a turbidity equivalent to a 0.5 McFarland Standard using a spectrophotometer at 530 nm, giving rise to a stock solution of 1×10⁶ cells/mL. Each yeast suspension was further diluted in growth medium to yield a final inoculum concentration of approximately 4×10³ cells/mL. Forty microlitres of the inoculum were used to inoculate each well, resulting in a final concentration of 2×10³ cells/mL. Drug-free controls were included on each plate. Plates were incubated for 16 hours at 32° C. The amount of cell growth was determined by particle density as read on a Nephelometer (BMG Lab Technologies; Durham, N.C.).

### Other Embodiments

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cellular and molecular biology, pharmacology, or related fields are intended to be within the scope of the invention.

What is claimed is:

1. A method for treating a fungal infection in a mammal in need thereof, said method comprising administering to the mammal (i) a compound having the formula: 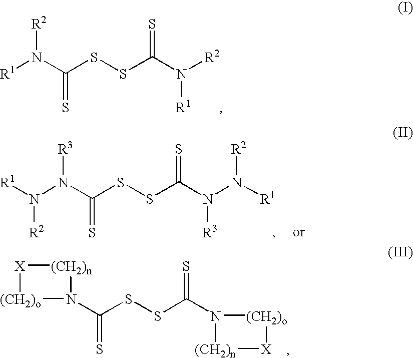
or pharmaceutically acceptable salts thereof, where x is CH₂, O, S, NR⁴, N(CH₂)ₚOR⁵, CH(CH₂)_{q}OR⁶, CH(CH₂)ᵣCO₂R⁷, CH(CH₂)_{S}CONR⁸R⁹, 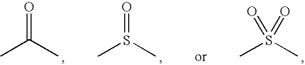
where R¹ and R² are independently c₁-C₈ linear or branched alkyl, alkaryl, or aryl, R³, R⁴, R⁵, R⁶, R⁷, R⁸, and R⁹ are independently H, C₁-C₈ linear or branched alkyl, alkaryl, or aryl, n is 0-3, o is 2-4, p is 2-6, and each q, r, or s is 0-6; and (ii) an antifungal agent,
wherein the compound of formula (i), (ii), or (iii) and the antifungal agent are administered simultaneously or within 14 days of each other, in amounts sufficient to treat said fungal infection.

2. The method of claim 1, wherein said fungal infection is an infection of Candida albicans or Candida glabrata.

3. The method of claim 1, wherein said fungal infection is an infection of Aspergillus fumigatus.

4. The method of claim 1, wherein said mammal is a human.

5. The method of claim 1, wherein said compound of formula (I), (II), or (III) and said antifungal agent are administered within ten days of each other.

6. The method of claim 5, wherein said compound of formula (I), (II), or (III) and said antifungal agent are administered within five days of each other.

7. The method of claim 6, wherein said compound of formula (I), (II), or (III) and said antifungal agent are administered within twenty-four hours of each other.

8. The method of claim 7, wherein said compound of formula (I), (II), or (III) and said antifungal agent are administered simultaneously.

9. The method of claim 1, wherein said antifungal agent is an ergosterol biosynthesis inhibitor.

10. The method of claim 9, wherein said antifungal agent is a polyene macrolide.

11. The method of claim 1, wherein said antifungal agent is an antimetabolite.

12. The method of claim 1, wherein said antifungal agent is a glucan synthesis inhibitor.

13. The method of claim 1, wherein said antifungal agent is amphotericin B, amorolfine, anidulafungin, butenafine, butoconazole, candidin, carbol-fuchsin, caspofungin, ciclopirox, clotrimazole, dapsone, econazole, emlkonazole, fluconazole, flucytosine, gentian violet, griseofulvin, haloprogin, itraconazole, ketoconazole, mafenide, micafungin, miconazole, naftifine, nystatin, oxiconazole, pimaricin, posaconazole, ravoconazole, rimocidin, silver sulfadiazine, sulconazole, terbinafine, terconazole, tioconazole, tolnaftate, undecylenic acid, vacidin A, or voriconazole.

14. The method of claim 1, wherein at least one of said compound of formula (I), (II), or (III) and said antifungal agent is administered systemically.

15. The method of claim 1, wherein at least one of said compound of formula (I), (II), or (III) and said antifungal agent is administered topically.

16. The method of claim 1, wherein at least one of said compound of formula (I), (II), or (III) and said antifungal agent is administered vaginally.

17. The method of claim 1, wherein at least one of said compound of formula (I), (II), or (III) and said antifungal agent is administered ophthalmically.

18. The method of claim 12, wherein said antifungal agent is amphotericin B, nystatin, candidin, rimocidin, vacidin A, or pimaricin.

19. The method of claim 18, wherein said antifungal agent is amphotericin B.

20. The method of claim 1, wherein said compound of formula (I), (II), or (III) is disulfiram (bis(diethylthiocarbamoyl) disulfide), bis(dimethylthiocarbamoyl) disulfide, bis(dipropylthiocarbamoyl) disulfide, bis(dibutylthiocarbamoyl) disulfide, bis(dipentylthiocarbamoyl) disulfide, bis(di(2-methylpropyl)thiocarbamoyl) disulfide, bis(piperidinothiocarbamoyl) disulfide, bis(morpholinothiocarbamoyl) disulfide, bis((4-methylpiperazino)thiocarbamoyl) disulfide, bis((4-(2-hydroxyethyl)piperazino)thiocarbamoyl) disulfide, bis((hexahydro-4-methyl-1H-1,4-diazepin-1-yl)thiocarbamoyl) disulfide, or bis(3,3-dimethylcarbazoyl) disulfide.

21. The method of claim 20, wherein said compound of formula (1), (II), or (III) is disulfiram.

22. The method of claim 1, wherein said antifungal agent is administered in an amount between 1 and 2000 milligrams and said compound of formula (I), (II), or (III) is administered in an amount between 1 and 1000 milligrams.

23. The method of claim 22, wherein said compound of formula (I), (II), or (III) is administered in an amount between 1 and 500 milligrams.

24. The method of claim 23, wherein said compound of formula (I), (II), or (III) is administered in an amount between 1 and 250 milligrams.

25. The method of claim 22, wherein said antifungal agent is present in an amount between 1 and 25 milligrams.

26. A pharmaceutical pack comprising an antifungal agent and a compound of formula (I), (II), or (III).

27. The pharmaceutical pack of claim 26 wherein the antifungal agent and said compound of formula (I), (II), or (III) are formulated separately and in individual dosage amounts.

28. The pharmaceutical pack of claim 26 wherein the antifungal agent and said compound of formula (I), (II), or (III) are formulated together.

29. A method for treating a mammal diagnosed with or at risk for developing a fungal infection, said method comprising administering to the patient (i) a low dosage of an antifungal agent and (ii) said compound of formula (I), (II), or (III), wherein the antifungal agent and said compound of formula (I), (II), or (III) are administered simultaneously or within 14 days of each other, in amounts sufficient to treat said mammal.

30. The method of claim 29, wherein said antifungal agent and said compound of formula (I), (II), or (III) are administered within ten days of each other.

31. The method of claim 30, wherein said antifungal agent and said compound of formula (I), (II), or (III) are administered within five days of each other.

32. The method of claim 31, wherein said antifungal agent and said compound of formula (I), (II), or (III) are administered within twenty-four hours of each other.

33. The method of claim 32, wherein said antifungal agent and said compound of formula (I), (II), or (III) are administered simultaneously.

34. The method of claim 29, wherein said antifungal agent is amphotericin B, amorolfine, anidulafungin, butenafine, butoconazole, carbol-fuchsin, caspofungin, ciclopirox, clotrimazole, dapsone, econazole, emlkonazole, fluconazole, flucytosine, gentian violet, griseofulvin, haloprogin, itraconazole, ketoconazole, mafenide, micafungin, miconazole, naftifine, nystatin, oxiconazole, pimaricin, posaconazole, ravoconazole, silver sulfadiazine, sulconazole, terbinafine, terconazole, tioconazole, tolnaftate, undecylenic acid, or voriconazole.

35. The method of claim 29, wherein said compound of formula (I), (II), or (III) is disulfiram (bis(diethylthiocarbamoyl) disulfide), bis(dimethylthiocarbamoyl) disulfide, bis(dipropylthiocarbamoyl) disulfide, bis(dibutylthiocarbamoyl) disulfide, bis(dipentylthiocarbamoyl) disulfide, bis(di(2-methylpropyl)thiocarbamoyl) disulfide, bis(piperidinothiocarbamoyl) disulfide, bis(morpholinothiocarbamoyl) disulfide, bis((4-methylpiperazino)thiocarbamoyl) disulfide, bis((4-(2-hydroxyethyl)piperazino)thiocarbamoyl) disulfide, or bis((hexahydro-4-methyl-1H-1,4-diazepin-1-yl)thiocarbamoyl) disulfide.

36. The method of claim 35, wherein said compound of formula (I), (II), or (III) is disulfiram.

37. The method of claim 36, wherein said antifungal agent is amphotericin B.

38. A method for identifying combinations of compounds useful for treating fungal infections in a patient in need of such treatment, said method comprising the steps of:
(a) contacting fungal cells or cells infected with a fungus in vitro with (i) a compound of formula (I), (II), or (III); and (ii) a candidate compound; and
(b) determining whether the combination of said compound of formula (I), (II), or (III) and said candidate compound reduces fungal growth relative to cells contacted with said compound of formula (I), (II), or (III) but not contacted with said candidate compound or cells contacted with said candidate compound but not with said compound of formula (I), (II), or (III), wherein a reduction of said fungal growth identifies said combination as a combination that is useful for treating a patient in need of such treatment.
